(19) United States
(12) Patent Application Publication
(10) Pub. No.: US 2007/0251527 A1
(43) Pub. Date: Nov. 1, 2007

(54) Self-contained respiratory therapy apparatus for enhanced patient compliance and therapeutic efficacy

(75) Inventor: Geoffrey P. Sleeper, Bay Village, OH (US)

Correspondence Address: BAKER & HOSTETLER LLP, WASHINGTON SQUARE, SUITE 1100, 1050 CONNECTICUT AVE. N.W., WASHINGTON, DC, 20036-5304, US

(73) Assignee: TIARA MEDICAL SYSTEMS, INC.

(21) Appl. No.: 11/785,993

(22) Filed: Apr. 23, 2007

Related U.S. Application Data
(60) Provisional application No. 60/745,378, filed on Apr. 21, 2006.

(51) Int. Cl. A61M 16/00; A62B 7/00

(52) U.S. Cl. 128/204.21; 128/204.18

(57) ABSTRACT

A self-contained respiratory apparatus, including a blower delivering a gas, a headgear integrated with the blower to support a respiratory interface, and a sensor embedded in the headgear and mounted to deliver data to a microprocessor integrated with the blower, the blower being controlled through the response of the sensor and the control of the microprocessor to support the respiratory interface through the headgear.

### CROSS-REFERENCE TO RELATED APPLICATIONS

This application claims priority to provisional U.S. patent application entitled, SELF-CONTAINED RESPIRATORY THERAPY APPARATUS FOR ENHANCED PATIENT COMPLIANCE AND THERAPEUTIC EFFICACY, filed Apr. 21, 2006, having a Ser. No. 60/745,378, the disclosure of which is hereby incorporated by reference in its entirety.

### FIELD OF THE INVENTION

The present invention relates generally to the field of respiratory therapy, and more particularly to a self-contained, adaptive system for delivery of a prescribed respiratory therapy.

### BACKGROUND OF THE INVENTION

Positive Airway Pressure (PAP) therapy, such as Continuous Positive Airway Pressure (CPAP) therapy, is a routinely prescribed treatment for certain respiratory conditions, such as Obstructive Apnea-Hypopnea Syndrome (OAHS). Briefly, OARS is a medical condition that involves a collapse in the patient's airway during sleep leading to numerous related conditions. Other names for OAHS and other related conditions include Obstructive Sleep Apnea (OSA), Obstructive Sleep Apnea Hypopnea Syndrome (OSAHS), Obstructive Sleep Apnea Syndrome (OSAS), Sleep Disordered Breathing (SDB) and others.

A typical PAP system includes a blower for producing the necessary positive air pressure, a nasal mask or other interface for delivering the air pressure to the patient's airway, a tube for connecting the blower to the mask, and headgear for supporting the mask and tube.

Patient compliance is a substantial factor in the effectiveness of PAP therapy in mitigating and/or preventing the occurrence of OARS. Numerous factors contribute to poor patient compliance, including: 1) discomfort related to a difficulty in adapting to the delivered pressure; 2) discomfort relating to the prescribed mask or other respiratory interface; and 3) discomfort resulting from an actual or perceived dryness or dehydration of the mucous membranes resulting from the therapy, especially in the patient's mouth and nasal passages.

One can define compliance in the 1980's, 6-8 h/d, was the expectation. A review of the current literature shows that the duration of CPAP use in compliant patients is 4-5 h/d. The current trend is actually lowered expectations for the patient compliance.

In the journal of “Behavioral Sleep Medicine”, there is included Dose-response relationship shown by Stepnowsky C J; Moore P J, “Nasal CPAP treatment for obstructive sleep apnea: developing a new perspective on dosing strategies and compliance.” J Psychosom Res, 2003; 54(6):599-605. Stepnowsky C J; Dimsdale J E. “Dose response relationship between CPAP compliance and measures of sleep apnea severity.” Sleep Med. 2002; 3(4):329-34.

Also, there is initial Experience in the home shown by Lewis K W, et al. “Early predictors of CPAP use for the treatment of obstructive sleep apnea.” Sleep. 2004; 27 (1):134-8.

Initial Experience during the titration night in the Sleep Lab is shown by Drake, et al. Sleep during titration predicts continuous positive airway pressure compliance.” Sleep. 2003; 26(3):308-11.

Initial Experience with attended PSG vs. Home based Autotitration is shown by Means M K, et al. “CPAP compliance in sleep apnea patients with and without laboratory CPAP titration.

There is also Cognitive-behavioral intervention, Aloia M S, et al. Improving compliance with nasal CPAP and vigilance in older adults with OAHS. Sleep Breath. 2001; 5(1):13-21 Video Instruction Wiese H, et al. CPAP compliance; video education may help! Sleep Med. 2005; 6(2): 171-4

The factors Contributing to Non-Compliance include the following:

1. Difficulty adapting to pressure,

2. Mask Discomfort

3. Nasal and Mouth Dryness

The CPAP Blowers can be the following:

1. “Blower in a box”

2. Bilevel;

3. Auto-titration; and

4. C-FLEX & EPR.

The “Blower in a Box” includes leak compensation and ramping. Bilevel Devices include PAP Devices capable of alternating pressure between I & E, Sense flow and/or pressure drop to drive cycle change.

The theory is to improve compliance with regard to Exhalation being more “comfortable”. There is also a need to increase compliance compared to conventional CPAP, as mentioned in Sanders M H, Kern N. OSA treated by independently adjusted inspiratory and expiratory positive airway pressures via nasal mask. Physiologic and clinical implications. Chest 1990 August; 98(2):317-24.

There is established efficacy of bilevel use in OSA as seen in the following:
1. Reeve-Hoche M K, Hudgel D W, et al. Continuous versus bilevel positive airway pressure for OSA. Am J Respir Crit Care Med. 1995 February; 151(2 Pt 1):443-9

62 patients over 1 year. No differences in compliance or symptoms/complaints
2. Gay P C, et al. A randomized, double blind clinical trial comparing CPAP with a novel bilevel pressure system for treatment of OSAS. Sleep 2003 November; 26(7):864-9
27 patients, randomized, controlled, double-blinded. No difference in compliance or clinical outcomes.

Auto-Titrating Devices are also referred to as auto-adjust CPAP. They use closed loop feedback and proprietary sensing and response algorithms.

Inputs for feedback/response include the following: Apnea, Obstructive hypopnea, Snoring/vibration, Flow limitation and Normal breathing.

The following articles show the above: Haniffa M; Lasserson T J; Smith I. Interventions to improve compliance with continuous positive airway pressure for obstructive sleep apnea. Cochrane database Syst Rev. 2004;(4):CDO03531

C-Flex & EPR are both proprietary systems utilize a pressure drop at exhalation. Both use an algorithm to detect the transition between inspiration and expiration. Both systems have three settings as shown by the following article: Aloia M S, et al. Treatment adherence and outcomes in flexible vs standard continuous positive airway pressure therapy. Chest 2005 June; 127(6):2085-93.

Eighty-nine patients (41 CPAP, 48 C-flex) were used in a study, and using sleepiness, other subjective scores, etc. equal between groups. C-flex users used device slightly longer than CPAP. Both groups device usage decreased with time.

The current masks have cause discomfort as shown in the known interfaces Nasal masks, full Full Face masks, Nasal pillows and cannula-style interfaces, and oral interfaces.

The known problems with the interfaces include Abrasions, Leaks, Claustrophobia (Chasens E R, et al. Claustrophobia and adherence to CPAP treatment. Western Journal of Nursing Research. 27(3):307-21, 2005 April), and Mouth breathing (Bachour A; Maasilta P. Mouth breathing compromises adherence to nasal CPAP therapy. Chest. 2004: 126(4):1248-54).

With regard to nasal and mouth dryness, compliance has been attempted by heating and humidifying blower air to help alleviate actual or perceived dryness.

Currently, much of the technology obtain only marginal increases in compliance. Both auto-titration and C-flex appear to be only benefiting a sub-set of CPAP users. Both appear to confer benefit by using algorithms to adjust pressures in response to sensors, which are very specific to use and the benefits are actually not conclusive.

Accordingly, it is desirable to provide increases in compliance that is not limited in the scope of coverage, bulky and inefficient in its application.

### SUMMARY OF THE INVENTION

The foregoing needs are met, to a great extent, by the present invention, wherein in one aspect an apparatus is provided that in some embodiments enhanced compliance and therapeutic efficacy based on a reduction of size of the apparatus for respiratory therapy.

In accordance with one embodiment of the present invention, the present invention includes a self-contained respiratory apparatus, including a blower delivering a gas, a headgear integrated with the blower to support a respiratory interface, and a sensor embedded in the headgear and mounted to deliver data to a microprocessor integrated with the blower, the blower being controlled through the response of the sensor and the control of the microprocessor to support the respiratory interface through the headgear.

In accordance with another embodiment of the present invention, the apparatus, includes a sensor collecting real-time data wirelessly, a blower connected to the sensor, a microprocessor using the real-time data collected from the sensor to control the blower to generate a minimum inspiratory pressure needed according to a predetermined criteria; and a user respiratory interface providing air from the blower to an area external from the apparatus, with the user interface being integrated with the blower.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a schematic illustration of a self-contained, adaptive respiratory apparatus worn by a patient according to an example embodiment of the present invention according to a preferred embodiment of the invention.

FIG. 2 is a top view of a blower of the apparatus in FIG. 1.

FIG. 3 is a back view of the blower of FIGS. 1 and 2.

FIG. 4 is a block diagram of the present invention.

### DETAILED DESCRIPTION

The invention will now be described with reference to the drawing figures, in which like reference numerals refer to like parts throughout.

To improve the compliance, one can reduce the mask/interface discomfort by incremental improvements, with adaptive, customized design, new materials, minimal size, and providing an integrated chinstrap as shown below by the present invention.

Physiologic customization can be beneficial, as evidenced by Series F; Marc, I. Importance of sleep stage and body position-dependence of sleep apnea in determining benefits to auto-CPAP therapy” Eur Respir. J. 2001; 18(1):170-5. The researchers concluded that some patients had sleep stage and body position-dependent obstruction breathing abnormalities. Ultra light sensors can be used for example with wireless technology. The sensors can collect real-time data. The sensors can collect real time data with respect to Oximetry, CO2, and EMG for REM detection.

The sensors do not have to be wireless, but can be placed in a remote position where the signal can be transmitted through either a wired or wireless method.

Microprocessor use real-time data to control the blower to generate the minimum therapeutic inspiratory pressure needed to provide therapeutic benefit.

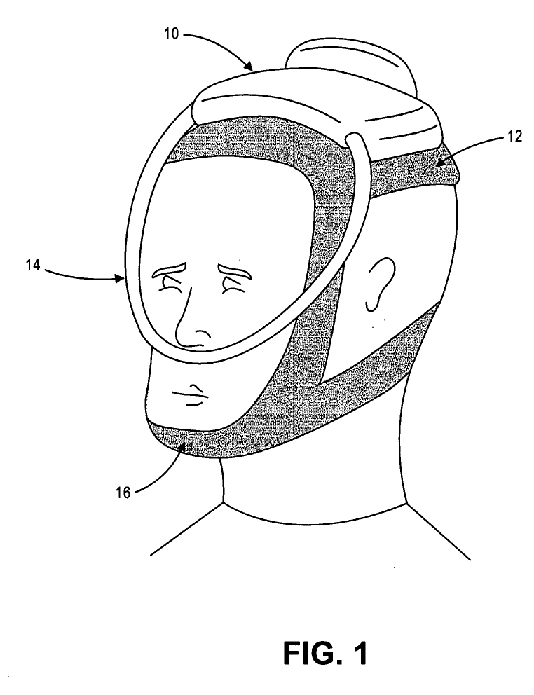

An embodiment of the present inventive apparatus is illustrated in FIG. 1. An example of an embodiment of the sensor includes Ultra-light miniature blower 10 being integrated into the headgear 12 that supports the patient respiratory interface 14.

Nanotechnology sensors can be embedded into the headgear 12, and/or are separately mounted and wireless connected to deliver data to the microprocessor, which is integrated into the blower 10.

The sensor is not limited to a wireless and nanotechnology sensor. Any other type of sensor can be used. For example, the sensor can have a wired connection or not be a nanotechnology sensor.

An integrated chinstrap 16 is provided on the headgear 12. An in-circuit HME is included to provide head and humidity to the air delivered by the blower 10. The HME stands for a know device of the heat and moisture exchanger.

The Ultra-light blower integrated into headgear provides: 1. High speed, very light low voltage motor blower (for example 12 volts); 2. Cushioning material between blower unit and headgear to reduce noise and vibration; 3. Micro processors imbedded either side to set and stabilize pressures 3-20 cm H2O; 4. Transformer on bed stand; 5. No need for 6 foot, 22 mm tube; and 6. Integrated nasal interface.

For purposes of the example embodiment or embodiments described herein, the present invention has been disclosed mainly in terms of its application to CPAP. Nevertheless, it is contemplated that the present invention can be applied to any appropriate form of respiratory therapy, including CPAP, VPAP (Variable Positive Airway Pressure) and BiPAP (Bi-level Positive Airway Pressure), Demand Positive Air Pressure (DPAP), numerous other forms of respiratory therapy, and any suitable combination thereof.

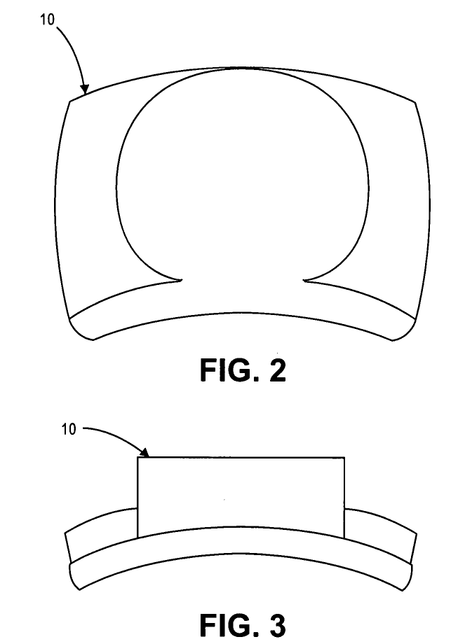

As seen in FIGS. 2-3, the blower 10 is shown in different views from the top and the back, respectively.

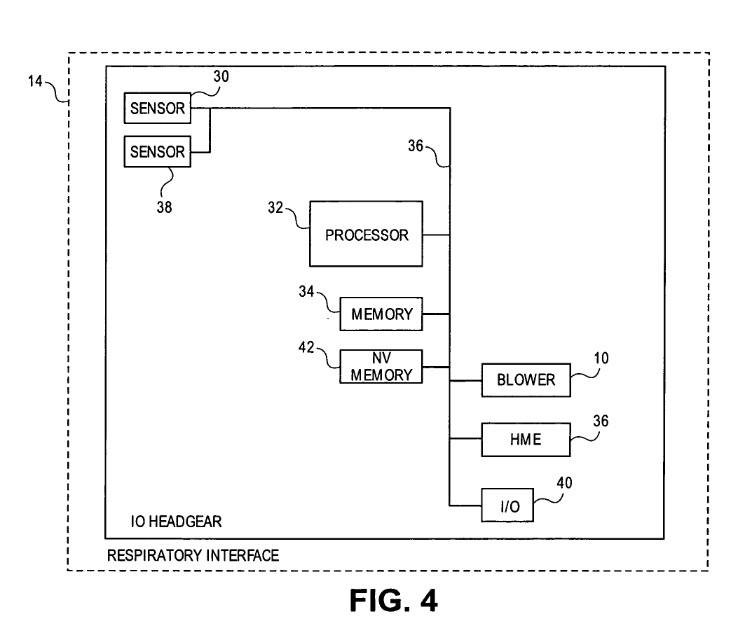

Referring to the block diagram of FIG. 4, the headgear 10 can include the microprocessor 32 being integrated into the headgear 10. The microprocessor can be connected on a master bus 36 with volatile memory 34 used to execute the instructions for the respiratory system, and non-volatile memory 42 for storing of information. The master bus 36 can also connect the processor 32 with the blower 10 for control of the blower 10 according to the instructions within the memory 34 and 42, and according to the feedback from the sensors 30 and 38. The connections to the transducers or sensors 30 and 38 can be wired or wireless with the processor 32. Wireless technologies can include for example BLUETOOTH or IEEE 802.11 standards for example. Furthermore, one or more of the sensors 30 and 38 can be also located outside of the headgear and not embedded within the headgear. The sensors 30 and 38 with give the feedback to the processor 32 for application of the system. An HME 36 can also be controlled by the processor 32 and input/output port 40 can be included for input or output of the data from the headgear 10. The headgear is then included within the respiratory interface 14. Therefore, as seen above, the integration of the headgear with the blower and other components vastly reduces the size and increases the efficiency.

Therefore, the ultralight blower 10 and housing with a remote sensing capacity can include the headgear 12 integrated with the chinstrap 16. There is also a continuous low profile of the nasal interface with the HME, and various nano-based or other type of physiological sensors can be included.

FIG. 4 is shown only as an example, the order and integration of the units can be changed. For example, as mentioned above, the sensors can be outside the headgear 10 or any other component. Additional components can also be added or removed. For example, the processor 32, and additionally the memory 34, 42 can be integrated into the blower unit 10 itself.

Further, it should be evident that this disclosure is by way of example and that various changes may be made by adding, modifying or eliminating details without departing from the fair scope of the teaching contained in this disclosure. The invention is therefore not limited to particular details of this disclosure.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. An apparatus, comprising:
a sensor collecting real-time data wirelessly;
a blower connected to the sensor;
a microprocessor using the real-time data collected from the sensor to control the blower to generate a minimum inspiratory pressure needed according to a predetermined criteria; and
a user respiratory interface, providing air from the blower to an area external from the apparatus, with the user interface being integrated with the blower.

2. The apparatus of claim 1, wherein the sensor, blower and microprocessor are integrated.

3. The apparatus of claim 1, further comprised of the user respiratory interface providing the air from the blower to the user, the user respiratory interface further comprising of a headgear attaching to a user, with the blower being integrated with the headgear.

4. The apparatus of claim 3, wherein the respiratory interface is integrated with the sensor, blower and microprocessor.

5. An apparatus, comprising:
a blower delivering a gas;
a headgear integrated with the blower to support a respiratory interface; and
a sensor embedded in the headgear and mounted to deliver data to a microprocessor integrated with the blower, the blower being controlled through the response of the sensor and the control of the microprocessor to support the respiratory interface through the headgear.

6. The apparatus of claim 5, further comprising an integrated chinstrap provided on the headgear.

7. The apparatus of claim 5, further comprising an in-circuit heat and moisture exchanger to provide heat and humidity to the gas delivered by the blower.

8. The apparatus of claim 5, further comprising a cushioning material between the blower and the headgear.

9. The apparatus of claim 5, wherein the microprocessor is embedded a certain side to set and stabilize pressure at certain pressures.

10. The apparatus of claim 5, further comprising of an integrated nasal interface to provide the gas from the blower.

11. The apparatus of claim 5, being used in any one of a continuous positive airway pressure, variable positive airway pressure, bi-level positive airway pressure, and demand positive air pressure therapy.

12. The apparatus of claim 5, being used in a positive airway pressure therapy.

13. The apparatus of claim 5, wherein the blower is integrated into the headgear.

14. The apparatus of claim 5, wherein the data collected by the sensor and transmitted to the microprocessor being approximately in real-time.

15. An apparatus, comprising:
a means for forcing a gas through a chamber;
a means for supporting a respiratory interface and integrated with the means for forcing the gas through the chamber; and
a transducing means embedded in the means for support and mounted to deliver data to a processing means integrated with the gas forcing means, the gas forcing means being controlled through the response of the transducing means and the control of the processing means to support the respiratory interface through the support means.

16. The apparatus of claim 15, further comprising an integrated chinstrap provided on the support means.

17. The apparatus of claim 15, further comprising an in-circuit heat and moisture exchanger means to provide heat and humidity to the gas delivered by the gas forcing means.

18. The apparatus of claim 15, further comprising a cushioning material between the gas forcing means and the support means.

19. The apparatus of claim 15, wherein the processing means is embedded a certain side to set and stabilize pressure at certain pressures.

20. The apparatus of claim 15, further comprising of an integrated nasal interface means to provide the gas from the gas forcing means.
